(19) United States
(12) Patent Application Publication
(10) Pub. No.: US 2019/0184142 A1
(43) Pub. Date: Jun. 20, 2019

(54) GUIDEWIRE ASSEMBLY WITH OFFSET CORE WIRES

(71) Applicant: Acclarent, Inc., Irvine, CA (US)

(72) Inventors: George L. Matlock, Pleasanton, CA (US); Don Q. Ngo-Chu, Irvine, CA (US); Tuan Pham, Huntington Beach, CA (US); John H. Thinnes, Mission Viejo, CA (US)

(21) Appl. No.: 15/842,089

(22) Filed: Dec. 14, 2017

(51) Int. Cl. A61M 25/09; A61B 17/24; A61B 34/20; A61B 34/10; A61M 29/02; A61B 90/30

(52) U.S. Cl. CPC A61M 25/09041; A61B 17/24; A61B 34/20; A61B 2090/365; A61M 29/02; A61B 90/30; A61B 2034/2051; A61B 34/10

(57) ABSTRACT

An apparatus and method of manufacture includes a helical wire coil body having a proximal body end portion and a distal body end portion that extend along a longitudinal coil axis. The apparatus also includes a non-extensible, core wire assembly configured to inhibit longitudinal elongation of the helical wire coil body along the longitudinal coil axis. The core wire assembly has a first core wire and a second core wire respectively extending from the proximal body end portion toward the distal body end portion. The second core wire proximally terminates relative to the first core wire such that the distal body end portion is more flexible than the proximal body end portion. The first and second core wires transversely overlap to provide a collective column strength to the helical wire coil body along the longitudinal coil axis.

### BACKGROUND

In some instances, it may be desirable to dilate an anatomical passageway in a patient. This may include dilation of ostia of paranasal sinuses (e.g., to treat sinusitis), dilation of the larynx, dilation of the Eustachian tube, dilation of other passageways within the ear, nose, or throat, etc. One method of dilating anatomical passageways includes using a guidewire and catheter to position an inflatable balloon within the anatomical passageway, then inflating the balloon with a fluid (e.g., saline) to dilate the anatomical passageway. For instance, the expandable balloon may be positioned within an ostium at a paranasal sinus and then be inflated, to thereby dilate the ostium by remodeling the bone adjacent to the ostium, without requiring incision of the mucosa or removal of any bone. The dilated ostium may then allow for improved drainage from and ventilation of the affected paranasal sinus. A system that may be used to perform such procedures may be provided in accordance with the teachings of U.S. Pub. No. 2011/0004057, entitled “Systems and Methods for Transnasal Dilation of Passageways in the Ear, Nose or Throat,” published Jan. 6, 2011, the disclosure of which is incorporated by reference herein. An example of such a system is the Relieva® Spin Balloon Sinuplasty™ System by Acclarent, Inc. of Irvine, Calif.

A variable direction view endoscope may be used with such a system to provide visualization within the anatomical passageway (e.g., the ear, nose, throat, paranasal sinuses, etc.) to position the balloon at desired locations. A variable direction view endoscope may enable viewing along a variety of transverse viewing angles without having to flex the shaft of the endoscope within the anatomical passageway. Such an endoscope that may be provided in accordance with the teachings of U.S. Pub. No. 2010/0030031, entitled “Swing Prism Endoscope,” published Feb. 4, 2010, the disclosure of which is incorporated by reference herein.

While a variable direction view endoscope may be used to provide visualization within the anatomical passageway, it may also be desirable to provide additional visual confirmation of the proper positioning of the balloon before inflating the balloon. This may be done using an illuminating guidewire. Such a guidewire may be positioned within the target area and then illuminated, with light projecting from the distal end of the guidewire. This light may illuminate the adjacent tissue (e.g., hypodermis, subdermis, etc.) and thus be visible to the naked eye from outside the patient through transcutaneous illumination. For instance, when the distal end is positioned in the maxillary sinus, the light may be visible through the patient's cheek. Using such external visualization to confirm the position of the guidewire, the balloon may then be advanced distally along the guidewire into position at the dilation site. Such an illuminating guidewire may be provided in accordance with the teachings of U.S. Pat. No. 9,155,492, entitled “Sinus Illumination Lightwire Device,” issued Oct. 13, 2015, the disclosure of which is incorporated by reference herein. An example of such an illuminating guidewire is the Relieva Luma Sentry™ Sinus Illumination System by Acclarent, Inc. of Irvine, Calif.

Image guided surgery (IGS) is a technique where a computer is used to obtain a real-time correlation of the location of an instrument that has been inserted into a patient's body to a set of preoperatively obtained images (e.g., a CT or Mill scan, 3-D map, etc.) so as to superimpose the current location of the instrument on the preoperatively obtained images. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) mounted thereon are used to perform the procedure while the sensors send data to the computer indicating the current position of each surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real time position of each surgical instrument relative to the anatomical structures shown in the scan images. In this manner, the surgeon is able to know the precise position of each sensor-equipped instrument by viewing the video monitor even if the surgeon is unable to directly visualize the instrument itself at its current location within the body.

Examples of electromagnetic IGS systems that may be used in ENT and sinus surgery include the Intertek ENT™ systems available from GE Medical Systems, Salt Lake City, Utah. Other examples of electromagnetic image guidance systems that may be modified for use in accordance with the present disclosure include but are not limited to the CARTO® 3 System by Bio sense-Webster, Inc., of Irvine, Calif.; systems available from Surgical Navigation Technologies,F Inc., of Louisville, Colo.; and systems available from Calypso Medical Technologies, Inc., of Seattle, Wash.

When applied to functional endoscopic sinus surgery (FESS), balloon sinuplasty, and/or other ENT procedures, the use of image guidance systems allows the surgeon to achieve more precise movement and positioning of the surgical instruments than can be achieved by viewing through an endoscope alone. This is so because a typical endoscopic image is a spatially limited, 2 dimensional, line-of-sight view. The use of image guidance systems provides a real time, 3-dimensional view of all of the anatomy surrounding the operative field, not just that which is actually visible in the spatially limited, 2 dimensional, direct line-of-sight endoscopic view. As a result, image guidance systems may be particularly useful during performance of FESS, balloon sinuplasty, and/or other ENT procedures where a section and/or irrigation source may be desirable, especially in cases where normal anatomical landmarks are not present or are difficult to visualize endoscopically.

While several systems and methods have been made and used in ENT procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a perspective view of an exemplary dilation instrument assembly, with an exemplary guidewire in a proximal position, and with a dilation catheter in a proximal position;

FIG. 1B depicts a perspective view of the dilation instrument assembly of FIG. 1A, with the guidewire in a distal position, and with the dilation catheter in the proximal position;

FIG. 1C depicts a perspective view of the dilation instrument assembly of FIG. 1A, with the guidewire in a distal position, with the dilation catheter in a distal position, and with a dilator of the dilation catheter in a non-dilated state;

FIG. 1D depicts a perspective view of the dilation instrument assembly of FIG. 1A, with the guidewire in a distal position, with the dilation catheter in the distal position, and with a dilator of the dilation catheter in a dilated state;

FIG. 2 depicts a schematic view of an exemplary image guided surgery (IGS) navigation system for use with the dilation instrument assembly of FIG. 1A;

FIG. 3 depicts a perspective view of a frame component of the image guided surgery navigation system of FIG. 2;

FIG. 4 depicts a perspective view of an exemplary medical procedure chair, with the frame component of the image guided surgery navigation system of FIG. 3 mounted to the chair;

FIG. 5 depicts a perspective view of a patient seated in the medical procedure chair of FIG. 4, with the image guided surgery navigation system of FIG. 2 being used to perform a procedure on the patient while seated in the chair;

FIG. 6 depicts a side elevational view of an exemplary illuminating guidewire for use in the dilation instrument assembly of FIG. 1A;

FIG. 7 depicts an enlarged side elevational view of the illuminating guidewire of FIG. 6;

FIG. 8 depicts an enlarged side cross-sectional view of the illuminating guidewire of FIG. 6 taken along a centerline thereof;

FIG. 9 depicts a side elevational view of an exemplary first alternative guidewire and a hub for use in the dilation instrument assembly of FIG. 1A with various features hidden for greater clarity of a core wire assembly;

FIG. 10 depicts an enlarged side elevational view of a distal portion of the core wire assembly of FIG. 9;

FIG. 11 depicts a cross-sectional view of the guidewire of FIG. 9 taken along section line 11-11 of FIG. 9;

FIG. 12 depicts a cross-sectional view of the guidewire of FIG. 9 taken along section line 12-12 of FIG. 9;

FIG. 13 depicts a cross-sectional view of the guidewire of FIG. 9 taken along section line 13-13 of FIG. 9; and

FIG. 14 depicts an enlarged side cross-sectional view of a distal portion of an exemplary second alternative guidewire, with a tethered navigation sensor, for use in the dilation instrument assembly of FIG. 1A.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

I. Overview of Exemplary Dilation Catheter System

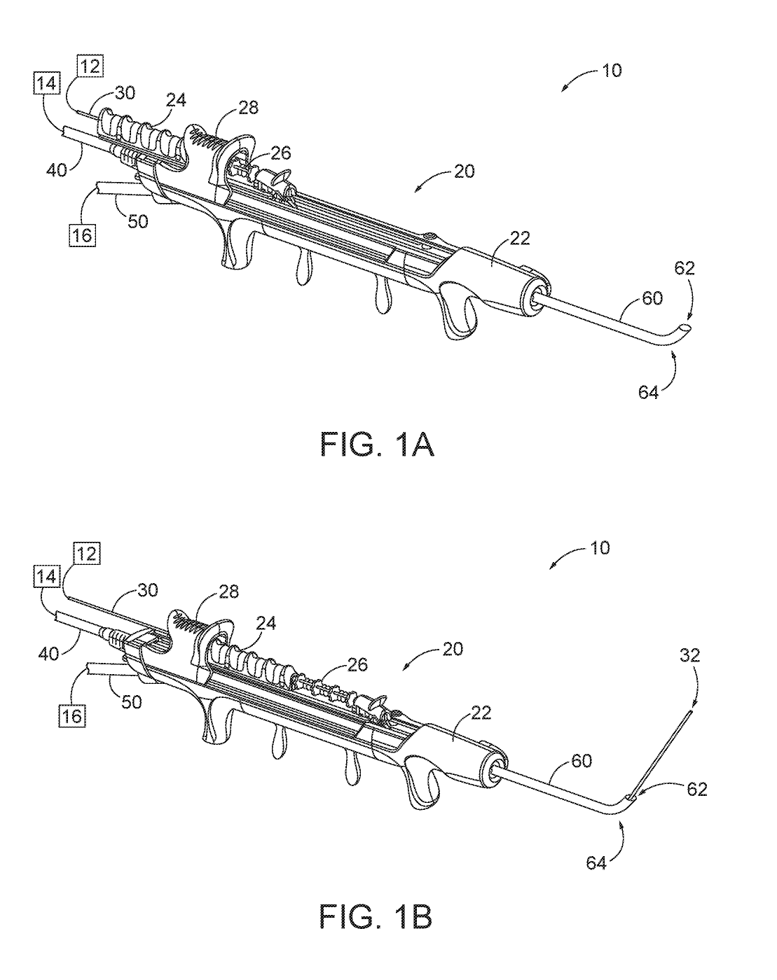

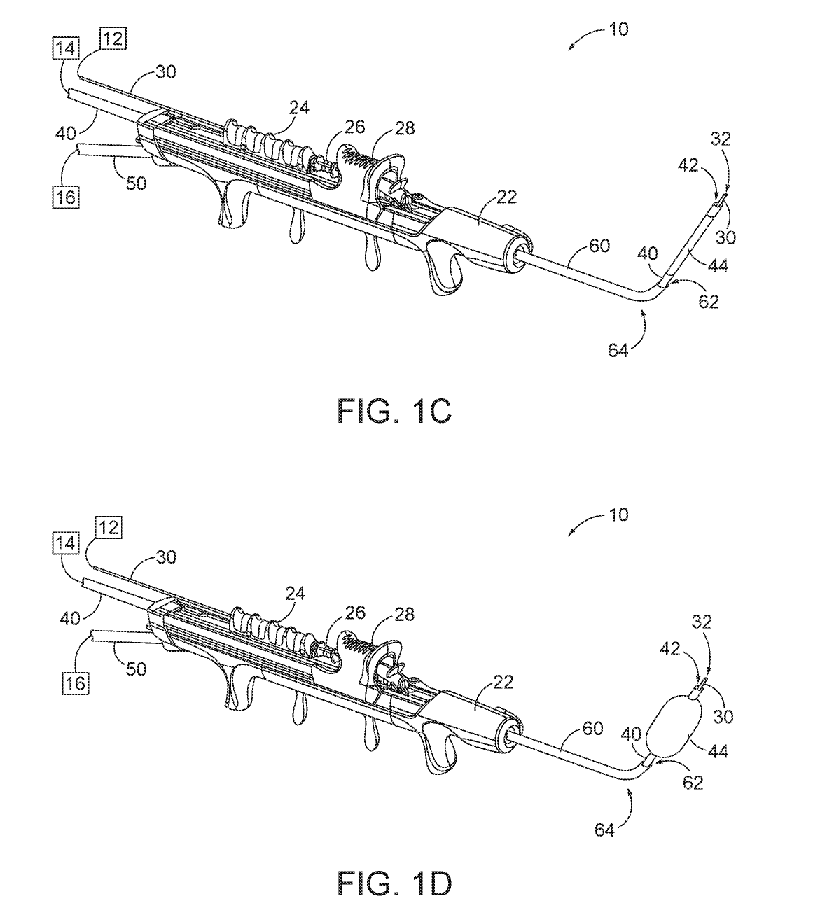

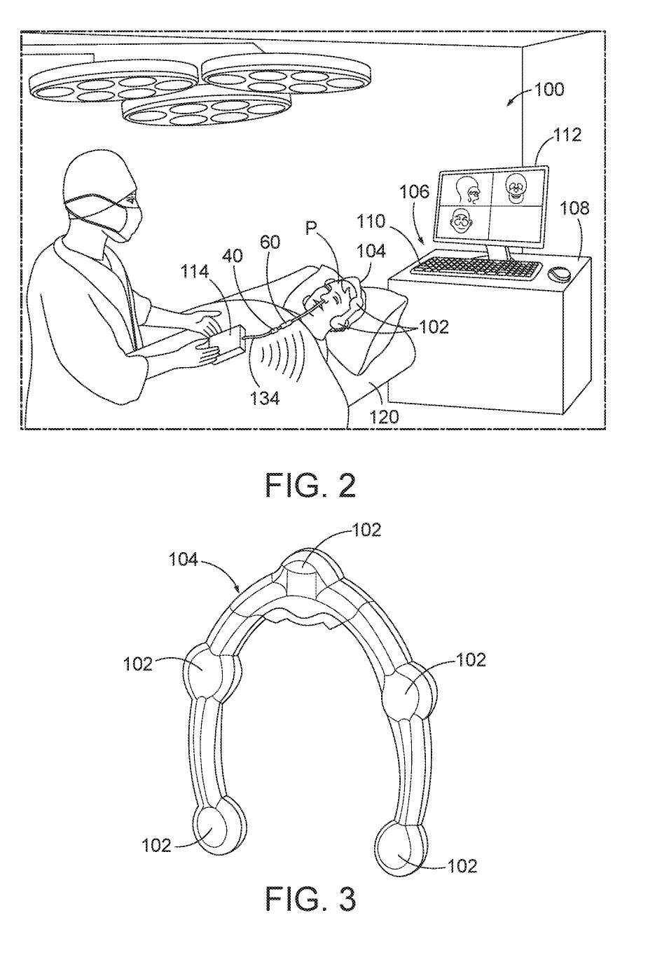

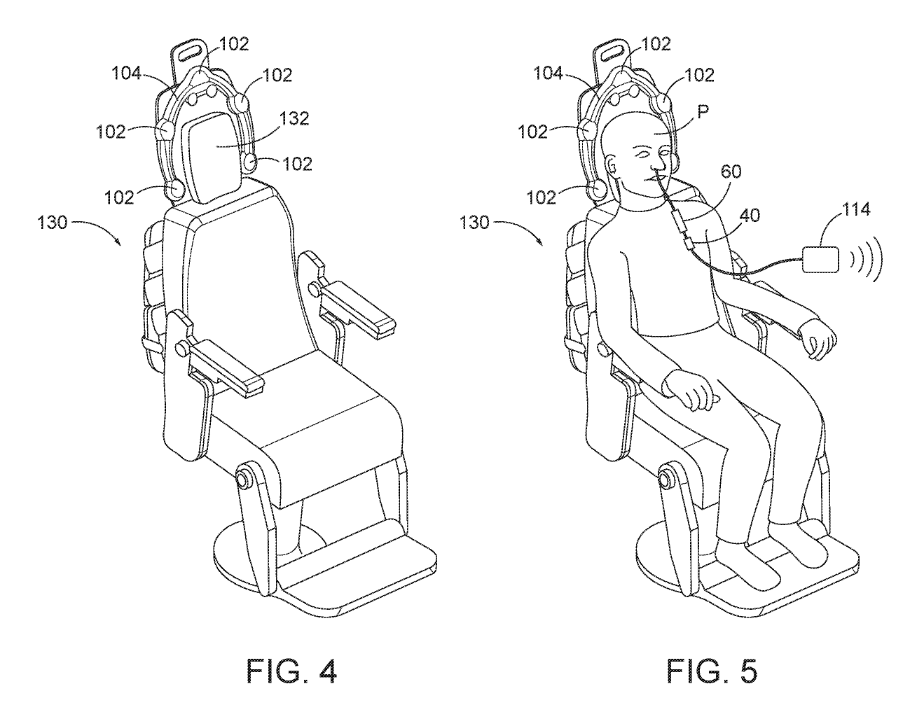

FIGS. 1A-1D shows a first exemplary dilation instrument assembly (10) that may be used to dilate the ostium of a paranasal sinus; to dilate some other passageway associated with drainage of a paranasal sinus; to dilate a Eustachian tube; or to dilate some other anatomical passageway (e.g., within the ear, nose, or throat, etc.). Dilation instrument assembly (10) of this example comprises a guidewire power source (12), an inflation source (14), an irrigation fluid source (16), and a dilation instrument (20). In some versions, guidewire power source (12) is part of an IGS system as described below with respect to FIGS. 2-3. In some other versions, guidewire power source (12) comprises a source of light as described below with respect to FIGS. 4-6. In the present example shown in FIGS. 1A-1D, inflation source (14) comprises a source of saline. However, it should be understood that any other suitable source of fluid (liquid or otherwise) may be used. Also in the present example, irrigation fluid source (16) comprises a source of saline. Again, though, any other suitable source of fluid may be used. It should also be understood that flush fluid source (16) may be omitted in some versions.

Dilation instrument (20) of the present example comprise a handle body (22) with a guidewire slider (24), a guidewire spinner (26), and a dilation catheter slider (28). Handle body (22) is sized and configured to be gripped by a single hand of a human operator. Sliders (24, 28) and spinner (26) are also positioned and configured to be manipulated by the same hand that grasps handle body (22). It should therefore be understood that dilation instrument (20) may be fully operated by a single hand of a human operator.

A. Exemplary Guide Catheter

A guide catheter (60) extends distally from handle body (22). Guide catheter (60) includes an open distal end (62) and a bend (64) formed proximal to open distal end (62). In the present example, dilation instrument (20) is configured to removably receive several different kinds of guide catheters (60), each guide catheter (60) having a different angle formed by bend (64). These different angles may facilitate access to different anatomical structures. Various examples of angles and associated anatomical structures are described in one or more of the references cited herein; while further examples will be apparent to those of ordinary skill in the art in view of the teachings herein. Guide catheter (60) of the present example is formed of a rigid material (e.g., rigid metal and/or rigid plastic, etc.), such that guide catheter (60) maintains a consistent configuration of bend (64) during use of dilation instrument (20). In some versions, dilation instrument (20), is further configured to enable rotation of guide catheter (60), relative to handle body (22), about the longitudinal axis of the straight proximal portion of guide catheter (60), thereby further promoting access to various anatomical structures.

B. Exemplary Guidewire

Dilation instrument (30) further comprises an exemplary guidewire (30), which is coaxially disposed in guide catheter (60). Guidewire slider (24) is secured to guidewire (30) such that translation of guidewire slider (24) relative to handle body (22) provides corresponding translation of guidewire (30) relative to handle body (22). In particular, translation of guidewire slider (24) from a proximal position (FIG. 1A) to a distal position (FIG. 1B) causes corresponding translation of guidewire (30) from a proximal position (FIG. 1A) to a distal position (FIG. 1B). When guidewire (30) is in a distal position, a distal portion of guidewire (30) protrudes distally from open distal end (62) of guide catheter (60). Guidewire spinner (26) is operable to rotate guidewire (30) about the longitudinal axis of guidewire (30). Guidewire spinner (26) is coupled with guidewire slider (24) such that guidewire spinner (26) translates longitudinally with guidewire slider (24).

In some versions, guidewire (30) includes a preformed bend formed just proximal to a distal end (32) of guidewire (30). In such versions, the preformed bend and the rotatability provided via guidewire spinner (26) may facilitate alignment and insertion of distal end (32) into a sinus ostium, Eustachian tube, or other passageway to be dilated. Also in some versions, guidewire (30) includes at least one optical fiber extending to a lens or other optically transmissive feature in distal end (32), such as illuminating guidewire (150) (see FIGS. 4-6) discussed below. Optical fiber may be in optical communication with guidewire power source (12), such that light may be communicated from guidewire power source (12) to distal end (32). In such versions, guidewire (30) may provide transillumination through a patient's skin in order to provide visual feedback to the operator indicating that distal end (32) has reached a targeted anatomical structure.

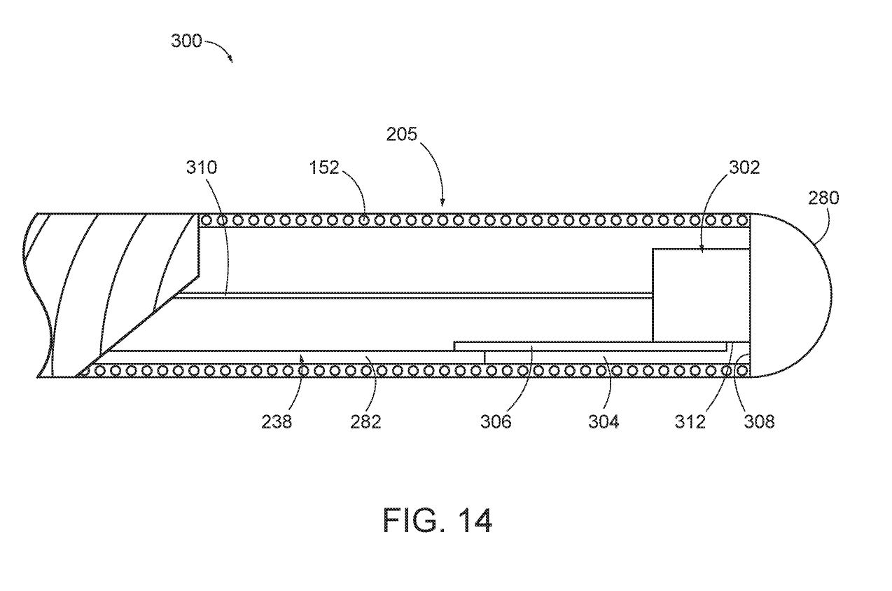

By way of example only, guidewire (30) may be configured in accordance with at least some of the teachings of U.S. Pat. No. 9,155,492, the disclosure of which is incorporated by reference herein. In some versions, guidewire (30) is configured similar to the Relieva Luma Sentry™ Sinus Illumination System by Acclarent, Inc. of Irvine, Calif. In addition to, or as an alternative to, including one or more optical fibers, guidewire (30) may include a sensor (302) (see FIG. 14) and at least one wire (310) (see FIG. 14) that enables guidewire (30) to provide compatibility with an IGS system as described in greater detail below. Other features and operabilities that may be incorporated into guidewire (30) will be apparent to those of ordinary skill in the art in view of the teachings herein.

C. Exemplary Dilation Catheter

Dilation instrument (30) further comprises a dilation catheter (40), which is coaxially disposed in guide catheter (60). Dilation catheter slider (28) is secured to dilation catheter (40) such that translation of dilation catheter slider (28) relative to handle body (22) provides corresponding translation of dilation catheter (40) relative to handle body (22). In particular, translation of dilation catheter slider (28) from a proximal position (FIG. 1B) to a distal position (FIG. 1C) causes corresponding translation of dilation catheter (40) from a proximal position (FIG. 1B) to a distal position (FIG. 1C). When dilation catheter (40) is in a distal position, a distal portion of dilation catheter (40) protrudes distally from open distal end (62) of guide catheter (60). As can also be seen in FIG. 1C, a distal portion of guidewire (30) protrudes distally from the open distal end of dilation catheter (40) when guidewire (30) and dilation catheter are both in distal positions.

Dilation catheter (40) of the present example comprises a non-extensible balloon (44) located just proximal to an open distal end (42) of dilation catheter (40). Balloon (44) is in fluid communication with inflation source (14). Inflation source (14) is configured to communicate fluid (e.g., saline, etc.) to and from balloon (44) to thereby transition balloon (44) between a non-inflated state and an inflated state. FIG. 1C shows balloon (44) in a non-inflated state. FIG. 1D shows balloon (44) in an inflated state. In some versions, inflation source (14) comprises a manually actuated source of pressurized fluid. In some such versions, the manually actuated source of pressurized fluid is configured and operable in accordance with at least some of the teachings of U.S. Pub. No. 2014/0074141, entitled “Inflator for Dilation of Anatomical Passageway,” published Mar. 13, 2014, the disclosure of which is incorporated by reference herein. Other suitable configurations that may be used to provide a source of pressurized fluid will be apparent to those of ordinary skill in the art in view of the teachings herein.

While not shown, it should be understood that dilation catheter (40) may include at least two separate lumens that are in fluid isolation relative to each other. One lumen may provide a path for fluid communication between balloon (44) and inflation source (14). The other lumen may provide a path to slidably receive guidewire (30).

While dilation catheter (40) of the present example is configured to transition between a non-dilated state and a dilated state based on the communication of fluid to and from balloon (44), it should be understood that dilation catheter (40) may include various other kinds of structures to serve as a dilator. By way of example only, balloon (44) may be replaced with a mechanical dilator in some other versions. Dilation catheter (40) may be constructed and operable in accordance with any of the various references cited herein. In some versions, dilator catheter (40) is configured and operable similar to the Relieva Ultirra™ Sinus Balloon Catheter by Acclarent, Inc. of Irvine, Calif. In some other versions, dilator catheter (40) is configured and operable similar to the Relieva Solo Pro™ Sinus Balloon Catheter by Acclarent, Inc. of Irvine, Calif. Other suitable variations of dilation catheter (40) will be apparent to those of ordinary skill in the art in view of the teachings herein.

D. Exemplary Irrigation Features

In some instances, it may be desirable to irrigate an anatomical site. For instance, it may be desirable to irrigate a paranasal sinus and nasal cavity after dilation catheter (40) has been used to dilate an ostium or other drainage passageway associated with the paranasal sinus. Such irrigation may be performed to flush out blood, etc. that may be present after the dilation procedure. In some such cases, guide catheter (60) may be allowed to remain in the patient while guidewire (30) and dilation catheter (40) are removed. A dedicated irrigation catheter (not shown) may then be inserted into guide catheter (60) and coupled with irrigation fluid source (16) via tube (50), to enable irrigation of the anatomical site in the patient. An example of an irrigation catheter that may be fed through guide catheter (60) to reach the irrigation site after removal of dilation catheter (60) is the Relieva Vortex® Sinus Irrigation Catheter by Acclarent, Inc. of Irvine, Calif. Another example of an irrigation catheter that may be fed through guide catheter (60) to reach the irrigation site after removal of dilation catheter (40) is the Relieva Ultirra® Sinus Irrigation Catheter by Acclarent, Inc. of Irvine, Calif.

In some other versions, dilation catheter (40) includes an additional irrigation lumen and an associated set of irrigation ports near distal end (42), such that dilation catheter (40) may be coupled with irrigation fluid source (16) via tube (50). Thus, a separate, dedicated irrigation catheter is not necessarily required in order to provide irrigation.

By way of example only, irrigation may be carried out in accordance with at least some of the teachings of U.S. Pat. No. 7,630,676, entitled “Methods, Devices and Systems for Treatment and/or Diagnosis of Disorders of the Ear, Nose and Throat,” issued Dec. 8, 2009, the disclosure of which is incorporated by reference herein. Of course, irrigation may be provided in the absence of a dilation procedure; and a dilation procedure may be completed without also including irrigation. It should therefore be understood that dilation fluid source (16) and tube (50) are merely optional.

E. Exemplary Variations

In the present example, guidewire (30) is coaxially disposed within dilation catheter (40), which is coaxially disposed within guide catheter (60). In some other versions, guide catheter (60) is omitted from dilation instrument (20). In some such versions, a malleable guide member is used to guide guidewire (30) and dilation catheter (40). In some such versions, guidewire (30) is omitted and dilation catheter (40) is slidably disposed about the exterior of the internal malleable guide member. In some other versions, guidewire (30) is slidably disposed about the exterior of the internal malleable guide member; and dilation catheter (40) is slidably disposed about the exterior of guidewire (30). In still other versions, guidewire (30) is slidably disposed within the interior of the malleable guide member; and dilation catheter (40) is slidably disposed about the exterior of the malleable guide member.

By way of example only, versions of dilation instrument (20) that include a malleable guide member may be constructed and operable in accordance with at least some of the teachings of U.S. Pub. No. 2016/0310714, entitled “Balloon Dilation System with Malleable Internal Guide,” published Oct. 27, 2016, the disclosure of which is incorporated by reference herein. As another merely illustrative example, versions of dilation instrument (20) that include a malleable guide member may be constructed and operable in accordance with at least some of the teachings of U.S. patent application Ser. No. 14/928,260, entitled “Apparatus for Bending Malleable Guide of Surgical Instrument,” filed Oct. 30, 2015, the disclosure of which is incorporated by reference herein; and/or U.S. Pub. No. 2012/0071857, entitled “Methods and Apparatus for Treating Disorders of the Sinuses,” published Mar. 22, 2012, the disclosure of which is incorporated by reference herein.

It should be understood that the variations of dilation instrument (20) described below in the context of an IGS system may be incorporated into versions of dilation instrument (20) having a malleable guide just like the variations of dilation instrument (20) described below in the context of an IGS system may be incorporated into versions of dilation instrument (20) having a rigid guide catheter (60).

Various examples below describe the use of an IGS system to provide navigation of instruments within a patient. In particular, various examples below describe how dilation instrument assembly (10) may be modified to incorporate IGS system features. However, it should also be understood that dilation instrument assembly (10) may be used in conjunction with conventional image guidance instruments, in addition to being used with IGS system components. For instance, dilation instrument assembly (10) may be used in conjunction with an endoscope, at least to provide initial positioning of guide catheter (60) in a patient. By way of example only, such an endoscope may be configured in accordance with at least some of the teachings of U.S. Pub. No. 2010/0030031, the disclosure of which is incorporated by reference herein. Other suitable kinds of endoscopes that may be used with the various versions of dilation instrument assembly (10) described herein will be apparent to those of ordinary skill in the art.

II. Exemplary Guidance of Dilation Catheter System

A. Image Guided Surgery Navigation System

FIG. 2 shows an exemplary image guided surgery (IGS) navigation system (100) configured to perform a Eustachian tube treatment procedure on a patient (P). As described in greater detail below, IGS navigation system (100) includes a computer used to obtain a real-time correlation of the location of an instrument that has been inserted into the patient's body, such as balloon dilation catheter (40), to a set of preoperatively obtained images (e.g., a CT or MRI scan, 3-D map, etc.) so as to superimpose the current location of the instrument on the preoperatively obtained images. In some instances, a digital tomographic scan (e.g., CT or MRI, 3-D map, etc.) of the operative field is obtained prior to surgery. A specially programmed computer is then used to convert the digital tomographic scan data into a digital map. During surgery, an instrument having one or more sensors (e.g., electromagnetic coils that emit electromagnetic fields and/or are responsive to externally generated electromagnetic fields) mounted thereon is used to perform the procedure while the sensors send data to the computer, indicating the current position of the surgical instrument. The computer correlates the data it receives from the instrument-mounted sensors with the digital map that was created from the preoperative tomographic scan. The tomographic scan images are displayed on a video monitor along with an indicator (e.g., cross hairs or an illuminated dot, etc.) showing the real-time position of the surgical instrument relative to the anatomical structures shown in the scan images. In this manner, the surgeon is able to know the precise position of the sensor-equipped instrument by viewing the video monitor, even if the surgeon is unable to directly visualize the instrument itself at its current location within the body.

IGS navigation system (100) incorporates balloon dilation catheter (40) described above, and may further incorporate a suitable guide catheter, such as guide catheter (60) described above. As described in greater detail below, IGS navigation system (100) is configured to implement a navigation sensor (not shown) of dilation catheter (40) to provide real-time location tracking of distal end of dilation catheter (40) within the patient (P) during a surgical procedure, and thereby facilitate accurate positioning of dilation catheter (40) within the patient (P). While IGS navigation system (100) is described below in connection with the positioning of balloon dilation catheter (40) and variations thereof within the Eustachian Tube, it will be appreciated that IGS navigation system (100) may also be employed in procedures for accessing and treating various other anatomical passageways of a patient with dilation catheter (40) and the variations thereof described below. While a navigation sensor is not shown in FIGS. 2-5, a navigation sensor (302) with an electrically connected wire (310) is shown in another alternative exemplary guidewire (300) in FIG. 14. It will be appreciated that the description of navigation sensor (not shown) provided with respect to FIGS. 2-5 may similarly apply to navigation sensor (302) and vice versa.

IGS navigation system (100) of the present example includes a set of magnetic field generators (102). Before a surgical procedure begins, field generators (102) are positioned about the head of the patient (P). As best shown in FIG. 3, in the present example field generators (102) arranged integrally within a frame (104) having a horseshoe-like shape and configured to be positioned about the patient's head. In the example of FIG. 2, patient (P) is positioned on a medical procedure table (120), and frame (104) is positioned above table (120) and about the patient's head. Frame (104) may be mounted to any suitable support structure (not shown), which may be coupled directly to medical procedure table (120) or provided independently from table (120), such as a floor-mounted stand. In other examples, frame (104) may be secured directly to the head of patient (P). It should be understood that field generators (102) may be positioned at various other suitable locations relative to patient (P), and on various other suitable structures.

FIGS. 4 and 5 show another exemplary implementation of IGS navigation system (100), in which patient (P) is seated in a medical procedure chair (130). Frame (104) is mounted to a headrest (132) of chair (130) such that frame (104) extends about the head of patient (P) when seated in chair (130). Medical procedure chair (130) may be configured according to one or more teachings of U.S. Patent App. No. 62/555,824, entitled “Apparatus to Secure Field Generating Device to Chair,” filed Sep. 8, 2017, the disclosure of which is incorporated by reference herein.

Field generators (102) of IGS navigation system (100) are operable to transmit alternating magnetic fields of different frequencies into a region in proximity to frame (104), and thereby generate an electromagnetic field in the region. In the present example, field generators (102) and frame (104) are arranged relative to the patient (P) such that the resulting electromagnetic field is formed about the patient's head. In other examples, field generators (102) and frame (104) may be suitably arranged in various other manners so as to generate an electromagnetic field about various other portions of the patient's body. Various suitable components that may be used to form and drive field generators (102) will be apparent to those of ordinary skill in the art in view of the teachings herein.

Field generators (102) enable tracking of the position of navigation sensor (not shown), and thus, distal end of balloon dilation catheter (40) with navigation sensor (not shown) therein, is tracked while moving through the electromagnetic field generated by field generators (102). In particular, as described in greater detail below, electromagnetic navigation sensor (not shown) of balloon dilation catheter (40) is configured to interact with the electromagnetic field and generate an electric signal in response to movement of sensor (not shown) through the electromagnetic field. Navigation sensor (not shown) then communicates this signal to a processor (106) of IGS navigation system (100). Processor (106), in turn, receives the signal and determines the three-dimensional location of navigation sensor (not shown), and catheter distal end at which sensor (not shown) is arranged, within the electromagnetic field and thus the patient.

Processor (106) of IGS navigation system (100) comprises a processing unit that communicates with one or more memories, and is configured to control field generators (102) and other elements of IGS navigation system (100). In the present example, processor (106) is mounted in a console (108), which comprises operating controls (110) that include a keypad and/or a pointing device such as a mouse or trackball. A physician uses operating controls (110) to interact with processor (106) while performing the surgical procedure. Processor (106) uses software stored in a memory of processor (106) to calibrate and operate system (100). Such operation includes driving field generators (102), processing data received from navigation sensor (not shown), processing data from operating controls (110), and driving display screen (112). The software may be downloaded to processor (106) in electronic form, over a network, for example, or it may, alternatively or additionally, be provided and/or stored on non-transitory tangible media, such as magnetic, optical, or electronic memory.

Processor (106) is further operable to provide video in real time via display screen (112), showing the position of distal end of balloon dilation catheter (40) in relation to a video camera image of the patient's head, a CT scan image of the patient's head, and/or a computer generated three-dimensional model of the anatomy within and adjacent to the patient's nasal cavity. Display screen (112) may display such images simultaneously and/or superimposed on each other. Moreover, display screen (112) may display such images during the surgical procedure. Such displayed images may also include graphical representations of instruments that are inserted in the patient's head, such as dilation catheter (40), such that the physician may view the virtual rendering of the instrument at its actual location in real time. Such graphical representations may look like the instrument or may be a much simpler representation such as a dot, crosshairs, etc. By way of example only, display screen (112) may provide images in accordance with at least some of the teachings of U.S. Pat. Pub. No. 2016/0008083, entitled “Guidewire Navigation for Sinuplasty,” published Jan. 14, 2016, the disclosure of which is incorporated by reference herein. In the event that the physician is simultaneously using an endoscope, the endoscopic image may also be provided on display screen (112). The images provided through display screen (112) may assist the physician in maneuvering and otherwise manipulating instruments within the patient's head.

Any suitable device may be used to generate a three-dimensional model of the internal anatomy of the portion of the patient's body (e.g., head) about which the electromagnetic field is generated and into which balloon dilation catheter (40) is to be inserted for conducting a treatment procedure. By way of example only, such a model may be generated in accordance with at least some of the teachings of U.S. Pat. Pub. No. 2016/0310042, entitled “System and Method to Map Structures of Nasal Cavity,” published Oct. 27, 2016, the disclosure of which is incorporated by reference herein. Still other suitable ways in which a three-dimensional anatomical model may be generated will be apparent to those of ordinary skill in the art in view of the teachings herein. It should also be understood that, regardless of how or where the three-dimensional model is generated, the model may be stored on console (108). Console (108) may thus render images of at least a portion of the model via display screen (112), and further render real-time video images of the position of distal end of dilation catheter (40) in relation to the model via display screen (112).

In addition to connecting with processor (106) and operating controls (110), console (108) may also connect with other elements of IGS navigation system (100). For instance, as shown in FIG. 2, a communication unit (114) may be coupled with balloon dilation catheter (40) via a wire (134). Communication unit (114) of this example is configured to provide wireless communication of data and other signals between console (108) and navigation sensor (not shown) of dilation catheter (40). In some versions, communication unit (114) simply communicates data or other signals from navigation sensor (not shown) to console (108) uni-directionally, without also communicating data or other signals from console (108). In some other versions, communication unit (114) provides bi-directional communication of data or other signals between navigation sensor (not shown) and console (108). While communication unit (114) of the present example couples with console (108) wirelessly, some other versions may provide wired coupling between communication unit (114) and console (108). Various other suitable features and functionality that may be incorporated into communication unit (114) will be apparent to those of ordinary skill in the art in view of the teachings herein.

In addition to, or in lieu of, having the components and operability described herein, IGS navigation system (100) may be constructed and operable in accordance with at least some of the teachings of U.S. Pat. No. 8,702,626, entitled “Guidewires for Performing Image Guided Procedures,” issued Apr. 22, 2014, the disclosure of which is incorporated by reference herein; U.S. Pat. No. 8,320,711, entitled “Anatomical Modeling from a 3-D Image and a Surface Mapping,” issued Nov. 27, 2012, the disclosure of which is incorporated by reference herein; U.S. Pat. No. 8,190,389, entitled “Adapter for Attaching Electromagnetic Image Guidance Components to a Medical Device,” issued May 29, 2012, the disclosure of which is incorporated by reference herein; U.S. Pat. No. 8,123,722, entitled “Devices, Systems and Methods for Treating Disorders of the Ear, Nose and Throat,” issued Feb. 28, 2012, the disclosure of which is incorporated by reference herein; and U.S. Pat. No. 7,720,521, entitled “Methods and Devices for Performing Procedures within the Ear, Nose, Throat and Paranasal Sinuses,” issued May 18, 2010, the disclosure of which is incorporated by reference herein.

Similarly, in addition to, or in lieu of, having the components and operability described herein, IGS navigation system (100) may be constructed and operable in accordance with at least some of the teachings of U.S. Pat. Pub. No. 2014/0364725, entitled “Systems and Methods for Performing Image Guided Procedures within the Ear, Nose, Throat and Paranasal Sinuses,” published Dec. 11, 2014, the disclosure of which is incorporated by reference herein; U.S. Pat. Pub. No. 2014/0200444, entitled “Guidewires for Performing Image Guided Procedures,” published Jul. 17, 2014, the disclosure of which is incorporated by reference herein; U.S. Pat. No. 9,198,736, entitled “Adapter for Attaching Electromagnetic Image Guidance Components to a Medical Device,” issued Dec. 1, 2015, the disclosure of which is incorporated by reference herein; U.S. Pat. Pub. No. 2011/0060214, entitled “Systems and Methods for Performing Image Guided Procedures within the Ear, Nose, Throat and Paranasal Sinuses,” published Mar. 10, 2011, the disclosure of which is incorporated by reference herein; U.S. Pat. No. 9,167,961, entitled “Methods and Apparatus for Treating Disorders of the Ear Nose and Throat,” issued Oct. 27, 2015, the disclosure of which is incorporated by reference herein; and U.S. Pat. Pub. No. 2007/0208252, entitled “Systems and Methods for Performing Image Guided Procedures within the Ear, Nose, Throat and Paranasal Sinuses,” published Sep. 6, 2007, the disclosure of which is incorporated by reference herein.

B. Illumination Guidance System

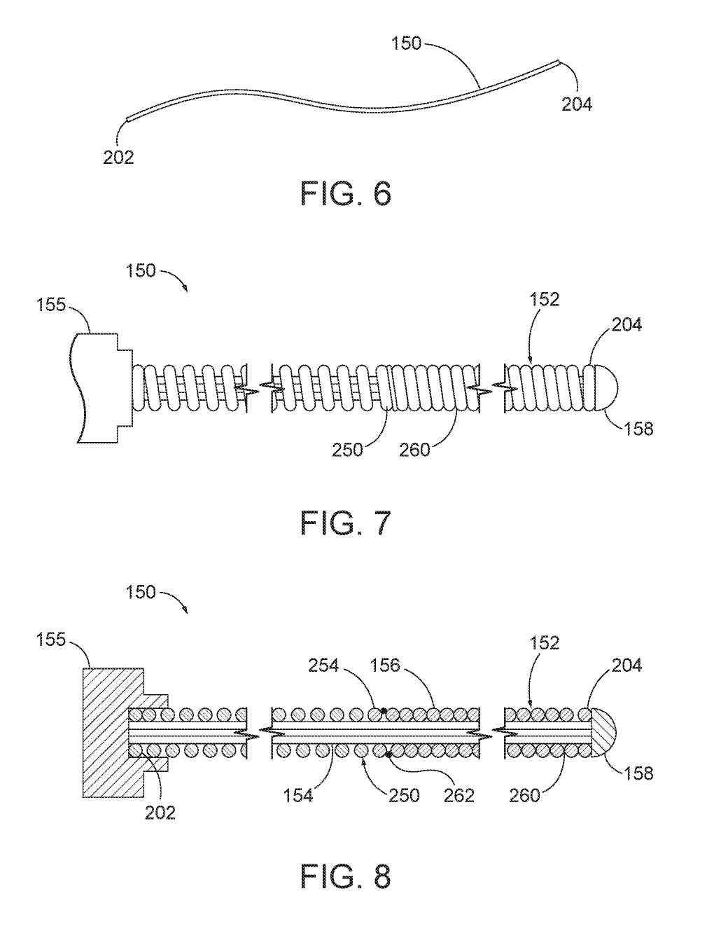

As shown in FIGS. 6-8, an exemplary illuminating guidewire (150) includes a coil body (152) positioned about a core wire (154). An illumination fiber (156) extends along the interior of core wire (154) and terminates in an atraumatic lens (158). A connector (155) at a proximal end of illuminating guidewire (150) enables optical coupling between illumination fiber (156) and a light source (not shown). Illumination fiber (156) may comprise one or more optical fibers. Lens (158) is configured to project light when illumination fiber (156) is illuminated by the light source, such that illumination fiber (156) transmits light from the light source to the lens (158). In some versions, a distal end of illuminating guidewire (150) is more flexible than the proximal end of illuminating guidewire (150). Illuminating guidewire (150) has a length enabling the distal end of illuminating guidewire (150) to be positioned distal to balloon (44) (see FIG. 1D) while the proximal end of illuminating guidewire (150) is positioned proximal to handle body (22) (see FIG. 1D). Illuminating guidewire (150) may include indicia along at least part of its length (e.g., the proximal portion) to provide the operator with visual feedback indicating the depth of insertion of illuminating guidewire (150) relative to dilation catheter (40) (see 1D). By way of example only, illuminating guidewire (150) may be configured in accordance with at least some of the teachings of U.S. Pub. No. 2012/0078118, the disclosure of which is incorporated by reference herein. In some versions, illuminating guidewire (150) is configured similar to the Relieva Luma Sentry™ Sinus Illumination System by Acclarent, Inc. of Irvine, Calif. Other suitable forms that illuminating guidewire (150) may take will be apparent to those of ordinary skill in the art in view of the teachings herein.

C. Distal End of Guidewire with Interlocking Coils

FIGS. 7-8 further show coil body (152) of guidewire (150) including a proximal end (202), a distal end (204), and an intermediate region (not shown) extending therebetween. Proximal end (202) and intermediate region (not shown) are generally constructed as discussed above in other examples provided herein. In addition, distal end (204) includes a proximal coil (250) of helical wire and a distal coil (260) of helical wire. A proximal end of proximal coil (250) proximally terminates in a solder joint (not shown), which joins a tubular member (not shown) with proximal coil (250). Proximal coil (250) helically extends from solder joint (not shown) to a distal end (254) of proximal coil and engages with a proximal end (262) distal coil (260). Coil body (152) of the present example is an assembly of two or more components, such as proximal and distal coils (250, 260). In an alternative example, coil body (152) may only have one such coil of wire. The term “coil body” as used herein may thus refer to a unitary structure or an assembly structure and is not intended to unnecessarily limit the invention. In some examples, proximal coil (250) may include a preformed bend (not shown) bent to an angle in accordance with bend angles known in the art of guidewires that are used in ENT surgical procedures.

Distal end (254) of proximal coil (250) and proximal end (262) of distal coil (260) are joined together in an interlocking fashion, such that the overlapping regions of coils (250, 260) form a double helix. More particularly, coils (250, 260) coaxially align along a longitudinal coil axis, which may be straight or bent as discussed above. By way of example only, the interlocking regions of ends (254, 262) may extend along approximately one to two full coil wraps of coils (250, 260). By way of further example only, the interlocking regions of ends (254, 262) may extend along a length between approximately 0.5 mm and approximately 0.75 mm. In the present example, proximal and distal coils (250, 260) are formed of metallic wires (e.g., stainless steel) wrapped in a helical configuration. Also in the present example, a ring of solder (not shown) is applied to the interlocking regions of coils (250, 260) to further secure the interlocking regions of coils (250, 260) together. By way of example only, ring of solder (not shown) may be formed of tin-silver solder. Alternatively, any other suitable material(s) may be used.

In the present example, coils (254, 262) have the same outer diameter but different inner diameters. By way of example only, coils (250, 260) may both have an outer diameter of approximately 0.0345 inches, with proximal coil (250) having an inner diameter of approximately 0.0225 inches, and with distal coil (260) having an inner diameter of approximately 0.0265 inches. Alternatively, any other suitable diameters may be used. Also in the present example, proximal coil (250) has a length of approximately 4.5 inches; while distal coil (260) has a length of approximately 4.25 mm. Alternatively, coils (250, 260) may have any other suitable lengths. Also in the present example, proximal coil (250) has an open pitch of approximately 0.75 mm, in which the open pitch of distal coil (260) is interlocked with a corresponding open pitch, though any other suitable pitch may be used. By way of further example only, the above-noted features of guidewire (150) may be constructed an operable in accordance with at least some of the teachings of U.S. Pat. App. No. 62/453,220, entitled “Navigation Guidewire with Interlocked Coils,” filed Feb. 1, 2017, the disclosure of which is incorporated by reference herein.

III. Exemplary Guidewire with Offset Core Wires

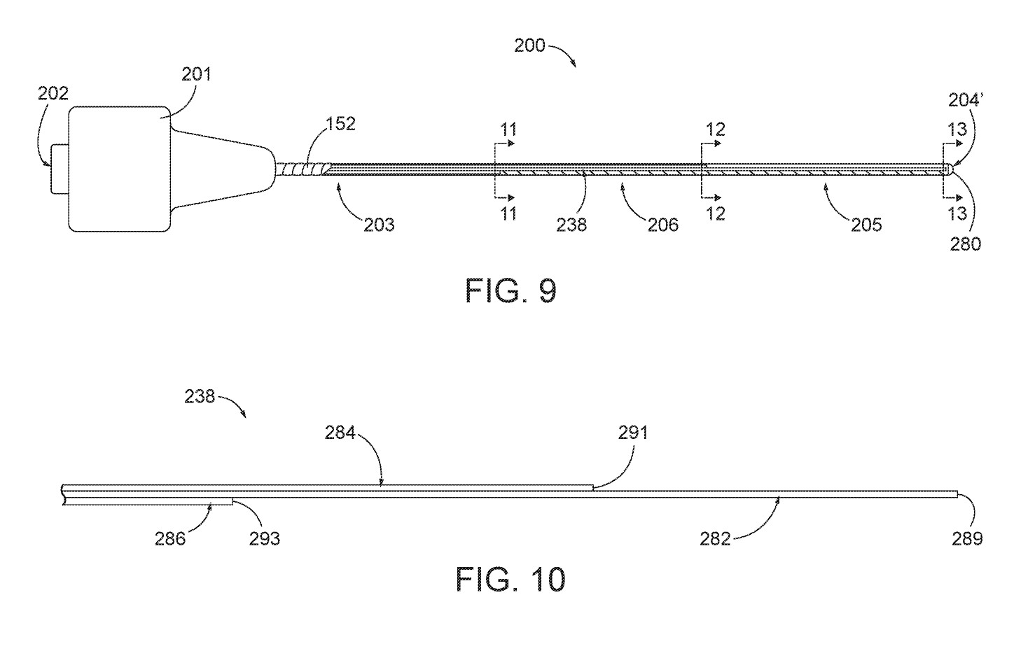

FIG. 9 shows an exemplary first alternative guidewire (200) that may be incorporated into dilation instrument assembly (10), in place of guidewire (30, 150). In some versions, at least a portion of the length of guidewire (200) (e.g., approximately 7 inches) is coated in one or more materials. By way of example only, at least a portion of the length of guidewire (200) may be coated in silicone. Other suitable materials that may be used as a coating for guidewire (200) will be apparent to those of ordinary skill in the art in view of the teachings herein. Except as otherwise described below, guidewire (200) is configured and operable similar to any one or more of the various guidewires (30, 150) described above. Guidewire (200) may be configured to provide IGS navigation system (100) compatibility or illumination guidance system compatibility to dilation instrument assembly (10).

Guidewire (200) of the present example extends from a hub (201) configured to removably connect to dilation instrument (20) (see FIG. 1A). Coil body (152) of guidewire (200) has a proximal end portion (203) with proximal end (202), a distal end portion (205) with a distal end (204′), and an intermediate portion (206) extending therebetween. Proximal end (202), intermediate portion (206), and distal end (204′) of coil body (152) are generally constructed as discussed above in other examples provided herein with like numbers indicating like features. However, rather than lens (158) (see FIG. 7) as discussed above with respect to distal end (204) (see FIG. 7) of illuminating guidewire (150) (see FIG. 7), distal end (204′) includes a tip member (280). Tip member (280) has an atraumatic, dome shape in the present example. In some versions, tip member (280) is formed by adhesive. In some other versions, tip member (280) is formed as a separate piece (e.g., of a polymer) and is then secured to distal coil (260), secured to adhesive, or secured to a sensor (not shown). Other suitable ways in which tip member (280) may be formed and secured will be apparent to those of ordinary skill in the art in view of the teachings herein.

With respect to FIGS. 9-10, guidewire (200) further includes a core wire assembly (238) within proximal and distal coils (250, 260) of coil body (152). Core wire assembly (238) is configured to inhibit longitudinal elongation of the proximal and distal coils (250, 260) along the longitudinal coil axis. To this end, core wire assembly (238) includes a plurality of core wires (282, 284, 286) configured to provide a collective column strength to coil body (152) while providing varying stiffness transverse to the longitudinal coil axis of coil body (152). In the present example, core wires (282, 284, 286) stiffen coil body (152) such that the distal end portion (250) of coil body (152) is the most flexible, the proximal end portion (203) of coil body (152) is the least flexible, and the intermediate portion (206) of coil body (152) is of medial flexibility between the distal and proximal end portions (205, 203). Thereby, the proximal, intermediate, and distal portions (203, 206, 205) of coil body (152) respectively and discretely increase in flexibility toward distal end (204′) for effective manipulation within the paranasal sinus and nasal cavity while simultaneously providing the collective column strength for insertion during use. As used herein, the term “stiffness” refers to the extent to which one or more portions of guide wire (200) resist deformation transverse to the longitudinal coil axis. In turn, the term “flexibility” refers to the complementary property of stiffness, such as being prone to deformation. Greater flexibility thus results in less stiffness and vice versa. To this extent, terms “stiffness” and “flexibility” are complementary, but otherwise interchangeable as used herein.

The present example of guide wire (200) has three core wires (282, 284, 286) and three discrete regions of flexibility extending respectively along proximal, intermediate, and distal portions (203, 206, 205) of coil body (152). Core wire assembly (238) includes long-length core wire (282), mid-length core wire (284), and short-length core wire (286) bundled together for collective column strength along the longitudinal coil axis and discrete flexibilities along the longitudinal coil axis. Core wires (282, 284, 286) are each formed of a non-extensible material that provides strength to the region of guidewire (200) along which core wires (282, 284, 286) extend. In particular, core wires (282, 284, 286) inhibit guidewire (200) from stretching longitudinally along the longitudinal coil axis. Moreover, other than the proximal and distal ends (202, 204′) of core wire assembly (238), the intermediate region of core wire assembly (238) is not fixedly secured within guidewire (200). Thus, core wire assembly (238) only affects flexibility as discussed below and does not adversely affect the lateral flexibility of guidewire (200).

Long-length core wire (282) extends from a proximal end (not shown) to a distal end (289) within distal end portion (205) of coil body (152). More particularly, distal end (289) of long-length core wire (282) extends to distal end of (204′) of coil body (152) adjacent to tip member (280). Mid-length core wire (284) extends from a proximal end (not shown) to a distal end (291) within intermediate portion (206) of coil body (152). Similarly, short-length core wire (284) extends from a proximal end (not shown) to a distal end (293) within proximal end portion (202) of coil body (152). In other words, distal end (291) of mid-length core wire (284) terminates proximally relative to distal end (289) of long-length core wire (282), whereas distal end (293) of short-length core wire (286) terminates proximally relative to distal end (291) of mid-length core wire (284). Each proximal end of core wires (282, 284, 286) respectively initiates at the same longitudinal position within a proximal end of hub (201) in the present example. More particularly, long-length core wire (282) is approximately 3.0 inches long, mid-length core wire (284) is approximately 2.2 inches long, and short-length core wire (286) is approximately 1.5 inches long. As described below in greater detail, the length differences between long-length, mid-length, and short-length core wires (282, 284, 286) effectively define the discrete regions of flexibility, such as high flexibility, medium flexibility, and low flexibility.

FIGS. 9-10 show the secured arrangement of core wires (282, 284, 286) and the staggered respective position of distal ends (289, 291, 293) for providing three discrete regions of high, medium, and low flexibility. Within proximal end portion (203) of coil body (152), core wires (282, 284, 286) of core wire assembly (238) overlap in a transverse direction relative to the longitudinal coil axis. Proximal ends of core wires (282, 284, 286) are secured within proximal end (202) of coil body (152) by a proximal end securement (not shown), which may be an overmolding, a soldering, a welding, an adhesive, an epoxy, or any other suitable means or techniques as will be apparent to those of ordinary skill in the art in view of the teachings herein. Accordingly, each core wire (282, 284, 286) respectively provides stiffness to the proximal end portion (203) to collectively define the region of low flexibility.

Distally beyond distal end (293) of short-length core wire (286) in intermediate portion (206), remaining core wires (282, 284) overlap in the transverse direction relative to the longitudinal coil axis to respectively provide stiffness without contributions from short-length core wire (286). Core wires (282, 284) without short-length core wire (286) thus collectively define the region of medium flexibility. Furthermore, distally beyond distal end (291) of mid-length core wire (284) in distal end portion (205), remaining core wire (282) provide stiffness without contributions from either one or both of short-length core wire (286) and mid-length core wire (284). Distal end (289) of core wire (282) is secured within distal end (204′) of coil body (152) by a distal end securement (296), which may be an overmolding, a soldering, a welding, an adhesive, an epoxy, or any other suitable means or techniques as will be apparent to those of ordinary skill in the art in view of the teachings herein. Core wire (282) alone thus defines the region of high flexibility.

In the present example, the region of low flexibility collectively defined by long-length core wire (282), mid-length core wire (284), and short-length core wire (286) is approximately 1.5 inches long, the region of medium flexibility collectively defined by long-length core wire (282) and mid-length core wire (284) is approximately 1.5 inches long, and the region of high flexibility defined by long-length core wire (282) is approximately 0.8 inches long. Alternative lengths of core wires and/or an alternative number core wires may be used in accordance with the invention described herein. For example, less than three core wires or more than three core wires of differing length may form an alternative core wire assembly (not shown). To this end, more core wires may be used to define additional regions of flexibility along the longitudinal coil axis to more particularly tune the flexibility of coil body (152) for a desired use. Such relative terms as “low,” “medium,” and “high,” with respect to flexibility are merely exemplary and not intended to limiting or absolute.

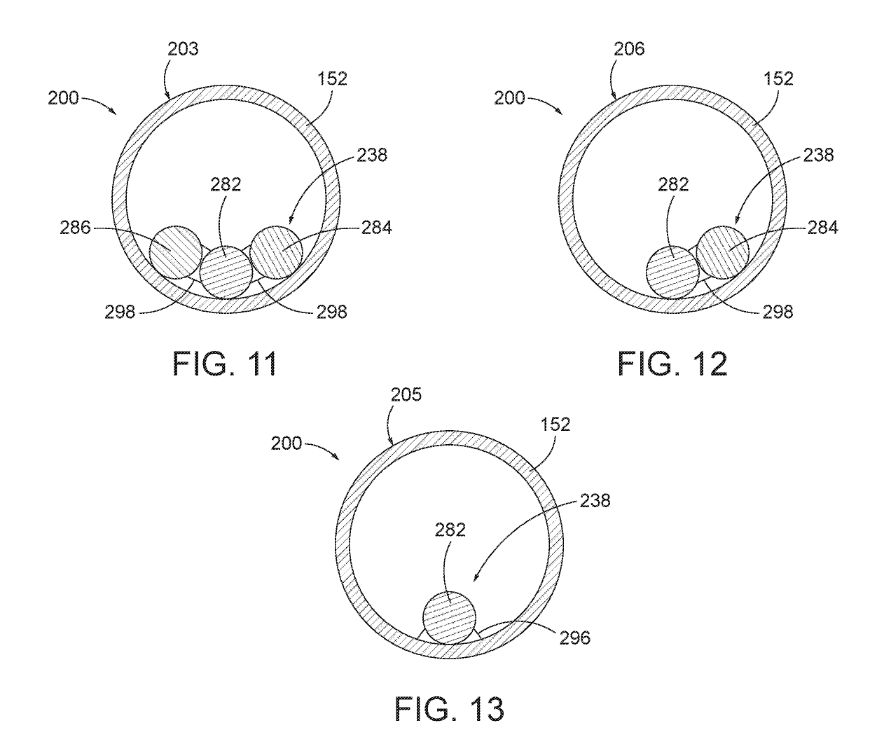

FIGS. 11-13 respectively show the arrangement of core wires (282, 284, 286) within proximal, intermediate, and distal portions (203, 206, 205) of guidewire (200) for low, medium, and high regions of flexibility along the longitudinal coil axis. As shown in each of FIGS. 11-13, core wires (282, 284, 286) extend in parallel with each other and with the longitudinal coil axis along coil body (152). In addition, the central axes defined by each core wire (282, 284, 286) are offset from each other such that the outer surfaces of each core wire (282, 284, 286) are secured together in contact. Long-length core wire (282) is centrally positioned between mid-length core wire (284) and short-length core wire (286) in order to secure each of mid-length and short-length core wires (284, 286) directly to long-length core wire (282) via a plurality of wire bundle securements (298). Wire bundle securements (298) respectively secure distal end (293) of short-length core wire (286) to long-length core wire (282) as well as distal end (291) of mid-length core wire (284) to long-length core wire (282). Additional wire bundle securements (298) are positioned along core wires (282, 284, 286) to further secure core wires (282, 284, 286) together with coil body (152). Such wire bundle securements (298) may be any combination of an overmolding, a soldering, a welding, an adhesive, an epoxy, or any other suitable means or techniques as will be apparent to those of ordinary skill in the art in view of the teachings herein. In one example, wire bundle securement is an overmolded polymer jacket around each of core wires (282, 284, 286). While the present example includes a side-by-side arrangement of core wires (282, 284, 286), it will be appreciated that core wires (282, 284, 286) may be alternatively arranged to overlap in the transverse direction for providing varying stiffness along the longitudinal coil axis. The invention is thus not intended to be unnecessarily limited to the arrangement of core wires (282, 284, 286) shown herein.

In manufacture, with respect to FIGS. 9-13, each of short-length and mid-length core wires (286, 284) is positioned in parallel with and against long-length core wire (282) in the side-by-side arrangement. Proximal ends of long-length, mid-length, and short-length core wires (282, 284, 286) longitudinally align, whereas distal ends (289, 291, 293) are longitudinally staggered. Proximal ends of short-length and mid-length core wires (286, 284) are longitudinally secured to proximal end of long-length core wire (282). Distal ends (293, 291) of short-length and mid-length core wires (286, 284) as secured to an outer surface of long-length core wire (282). Additional wire bundles securements (298) may be added between long-length core wire (282) and short-length and mid-length core wires (286, 284) as desired. Thereby, core wires (282, 284, 286) form core wire assembly (238) in the present example.

Core wire assembly (238) is inserted through coil body (152) such that proximal end (202) of coil body (152) longitudinally aligns with proximal ends of long-length, mid-length, and short-length core wires (282, 284, 286). Similarly, distal end (204′) of coil body (152) also aligns with distal end (289) of long-length core wire (282). Proximal end (202) of coil body (152) is secured to proximal ends of long-length, mid-length, and short-length core wires (282, 284, 286) by proximal end securement (not shown), and distal end (204′) of coil body (152) is secured to distal end (289) of long-length core wire (282) to form guidewire (200). Hub (201) is further connected to proximal end portion (203) for releasably coupling guide wire (200) to dilation instrument (20) (see FIG. 1A).

IV. Exemplary Guidewire with a Tethered Navigation Sensor

FIG. 14 shows an exemplary second alternative guidewire (300) having a tethered navigation sensor (302) that may be incorporated into dilation instrument assembly (10), in place of guidewire (30, 150, 200). Guidewire (300) includes core wire assembly (238) extending through coil body (152) as discussed above and, to this end, like numbers indicate like features. As shown particularly with respect to guidewire (200), core wire assembly (238) extends distally toward tip (280), but terminates proximally from tip (280) to define a gap (304) therebetween to maintain a relatively radially compact distal end portion (205) adjacent to tip (280). In one example, a tether (306) connected to long-length core wire (282) of core wire assembly (238) extends distally therefrom and connects to sensor (302) for inhibiting elongation of coil body (152) along gap (304) while maintaining the flexibility of coil body (152) as well as the relative compactness of distal end portion (205). While tether (306) is connected core to wire (282) in this example, alternative core wires (not shown) that are configured to provide the above discussed structural characteristics to coil body (152) may be similarly used with tether (306). The invention is thus not intended to be unnecessarily limited to use with core wire (282). Additional aspects of tethered navigation sensor (302) and guidewire (300) may be provided in accordance with the teachings of U.S. Pub. No. 2016/0310041, entitled “Guidewire with Navigation sensor,” published Oct. 27, 2016, the disclosure of which is incorporated by reference herein.

Tethered navigation sensor (302) is attached to a proximal face (308) of tip (280), and a wire (310) electrically connects tethered navigation sensor (302) to a remainder of IGS navigation system (100) for use. Tether (306) extends from long-length core wire (282) and attaches to a radial sidewall (312) of tethered navigation sensor (302) between radial sidewall (312) and coil body (152). In the present example, tether (306) is formed from a non-extensible material to limit gap (304) between long-length core wire (282) and tethered navigation sensor (302) to less than or equal to a predetermined distance, regardless of any flexing of distal end portion (205). Limiting such elongation provides a more durable connection between wire (310) and tethered navigation sensor (302) to increase the useful life of guidewire (300). Furthermore, tether (306) is sized to fit between tethered navigation sensor (302) and coil body (152) such that distal end portion (205) of coil body (152) surrounding tethered navigation sensor (302) defines a distal radial diameter less than or equal to the proximal portions of coil body (152) adjacent thereto. In other words, the size of tether (306) provides for the relatively radially compact distal end portion (205) adjacent to tip (280) as discussed briefly above.

V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### EXAMPLE 1

An apparatus, comprising: (a) a helical wire coil body extending along a longitudinal coil axis and including: (i) a proximal body end portion, and (ii) a distal body end portion; and (b) a non-extensible, core wire assembly configured to inhibit longitudinal elongation of the helical wire coil body along the longitudinal coil axis, wherein the core wire assembly includes: (i) a first core wire distally extending from the proximal body end portion toward the distal body end portion, and (ii) a second core wire distally extending from the proximal body end portion toward the distal body end portion and proximally terminating relative to the first core wire such that the distal body end portion is more flexible than the proximal body end portion, wherein the first core wire and the second core wire transversely overlap to provide a collective column strength to the helical wire coil body along the longitudinal coil axis.

### EXAMPLE 2

The apparatus of Example 1, wherein the helical wire coil body further includes an intermediate body portion extending between the proximal and distal body end portions, wherein the core wire assembly further includes a third core wire distally extending from the proximal body end portion toward the distal body end portion and proximally terminating relative to the second core wire such that the intermediate body portion is more flexible than the proximal body end portion, and wherein the first core wire, the second core wire, and the third core wire transversely overlap to provide the collective column strength to the helical wire coil body along the longitudinal coil axis.

### EXAMPLE 3

The apparatus of Example 2, wherein the intermediate body portion is less flexible than the distal body end portion.

### EXAMPLE 4

The apparatus of Example 3, wherein the first core wire terminates at a first distal wire end positioned within the distal body end portion, and wherein the second core wire terminates at a second distal wire end positioned within the intermediate body portion.

### EXAMPLE 5

The apparatus of Example 4, wherein the third core wire terminates at a third distal wire end positioned within the proximal body end portion.

### EXAMPLE 6

The apparatus of Example 5, wherein the second distal wire end proximally terminates from the first distal wire end with a distal length therebetween, and wherein the distal length therebetween is approximately 0.8 inches.

### EXAMPLE 7

The apparatus of Example 6, wherein the third distal wire end proximally terminates from the first distal wire end with a proximal length therebetween, and wherein the proximal length therebetween is approximately 1.5 inches.

### EXAMPLE 8

The apparatus of any one or more of Examples 1 through 7, wherein the second core wire is transversely secured relative to the first core wire by a wire bundle securement.

### EXAMPLE 9

The apparatus of Example 8, wherein the wire bundle securement comprises an overmolding.

### EXAMPLE 10

The apparatus of any one or more of Examples 8 through 9, wherein the core wire assembly has a distal tip, wherein the distal tip of the core wire assembly is secured to the distal body end portion of the helical wire coil body by a distal securement.

### EXAMPLE 11

The apparatus of any one or more of Examples 1 through 10, wherein the second core wire extends in parallel with the first core wire.

### EXAMPLE 12

The apparatus of Example 11, wherein the second core wire is positioned against the first core wire.

### EXAMPLE 13

The apparatus of any one or more of Examples 1 through 12, further comprising a navigation sensor and a non-extensible tether, wherein the distal body end portion of the helical wire coil body contains the navigation sensor therein, and wherein the tether extends from the core wire assembly to the navigation sensor and is configured to inhibit elongation of the navigation sensor relative to the core wire assembly.

### EXAMPLE 14

The apparatus of any one or more of Examples 1 through 13, wherein the helical wire coil body further includes: (i) a proximal wire coil, wherein the proximal wire coil is helical, and (ii) a distal wire coil, wherein the distal wire coil is helical and interlocked with the proximal wire coil such that the proximal and distal wire coils form a double helix configuration extending along the longitudinal coil axis.

### EXAMPLE 15

The apparatus of any one or more of Examples 1 through 14, further comprising: (a) a body; (b) a guide extending distally from the body; (c) a guidewire including the helical wire coil and the core wire assembly, wherein the guidewire is slidably disposed relative to the guide; and (d) a dilation catheter slidably disposed relative to the guidewire, wherein the dilation catheter includes an expandable dilator.

### EXAMPLE 16

An apparatus, comprising: (a) a helical wire coil body extending along a longitudinal coil axis and including: (i) a proximal body end portion, (ii) a distal body end portion having: (A) a proximal wire coil, wherein the proximal wire coil is helical, and (B) a distal wire coil, wherein the distal wire coil is helical and interlocked with the proximal wire coil such that the proximal and distal wire coils form a double helix configuration extending along the longitudinal coil axis, and (C) an intermediate body portion extending between the proximal and distal body end portions; and (b) a non-extensible, core wire assembly configured to inhibit longitudinal elongation of the helical wire coil body along the longitudinal coil axis, wherein the core wire assembly includes: (i) a first core wire distally extending from the proximal body end portion toward the distal body end portion, (ii) a second core wire distally extending from the proximal body end portion toward the distal body end portion and proximally terminating relative to the first core wire such that the distal body end portion is more flexible than the proximal body end portion, and (iii) a third core wire distally extending from the proximal body end portion toward the distal body end portion and proximally terminating relative to the second core wire such that the intermediate body portion is more flexible than the proximal body end portion, wherein the first core wire, the second core wire, and the third core wire extend in parallel with each other and transversely overlap to provide a collective column strength to the helical wire coil body along the longitudinal coil axis.

### EXAMPLE 17

The apparatus of Example 16, wherein the second core wire and the third core wire are transversely secured relative to the first core wire by a wire bundle securement.

### EXAMPLE 18

The apparatus of Example 17, wherein the second core wire and the third core wire are each respectively positioned against the first core wire.

### EXAMPLE 19

A method of manufacturing a guidewire, the method comprising: (a) securing a first core wire having a first wire length relative to a second core wire having a second wire length to form a non-extensible, core wire assembly, wherein the first wire length is longer than the second wire length; (b) inserting the core wire assembly through a helical wire coil body, wherein the helical wire coil body has a proximal body end portion and a distal body end portion and extends along a longitudinal coil axis; and (c) securing the core wire assembly within the helical wire coil body such that the helical wire coil body is non-extensible with a collective column strength along the longitudinal coil axis and the distal body end portion is more flexible than the proximal body end portion.

### EXAMPLE 20

The method of Example 19, wherein securing the first core wire having the first wire length relative to the second core wire having the second wire length further includes securing the first core wire relative to third second core wire having a third wire length to form the core wire assembly.

### EXAMPLE 21

The apparatus of any one or more of Examples 1 through 14, further comprising a dilation catheter including an expandable dilator.

### EXAMPLE 22

The apparatus of Example 21, wherein the dilation catheter is slidably disposed relative to the helical wire coil body and the core wire assembly.

### EXAMPLE 23

The apparatus of any one or more of Examples 21 through 22, wherein the expandable dilator includes an inflatable balloon configured to expand from a non-inflated state to an inflated state.

### EXAMPLE 24

A method of dilating an anatomical passageway with a surgical instrument including a guidewire and a dilation catheter having an expandable dilator, wherein the guidewire includes a helical wire coil body and a non-extensible, core wire assembly, wherein the helical wire coil body has a proximal body end portion and a distal body end portion, wherein the core wire assembly is configured to inhibit longitudinal elongation of the helical wire coil body along the longitudinal coil axis, wherein the core wire assembly includes a first core wire and a second core wire, wherein the first core wire distally extends from the proximal body end portion toward the distal body end portion, wherein the second core wire distally extends from the proximal body end portion toward the distal body end portion and proximally terminates relative to the first core wire such that the distal body end portion is more flexible than the proximal body end portion, and wherein the first core wire and the second core wire transversely overlap to provide a collective column strength to the helical wire coil body along the longitudinal coil axis, the method comprising: (a) inserting the guidewire into the anatomical passageway; (b) guiding the dilation catheter along the guidewire through the anatomical passageway; and (c) expanding the dilator to an expanded state against an anatomy about the anatomical passageway to dilate the anatomical passageway.

### EXAMPLE 25

The method of Example 24 wherein inserting the guidewire into the anatomical passageway further includes providing a collective column strength to the helical wire coil body along the longitudinal coil axis with the first and second core wires.

### EXAMPLE 26

The method of any one or more of Examples 24 through 25 further comprising generating an image of at least a portion of the anatomical passageway.

### EXAMPLE 27

The method of any one or more of Examples 24 through 26 wherein at least one of the guidewire or the dilation catheter includes a navigation sensor, the method further comprising tracking a position of the navigation sensor.

### EXAMPLE 28

The method of Example 7 wherein tracking the position of the navigation sensor further includes generating an electromagnetic field about the anatomical passageway to detect the position of the navigation sensor.

### EXAMPLE 29

The method of any one or more of Examples 24 through 28 wherein the guidewire further includes an optically transmissive feature, the method further comprising providing visual feedback to an operator indicating a position of the guidewire within the anatomical passageway.

### EXAMPLE 30

An apparatus, comprising: (a) a helical wire coil body extending along a longitudinal coil axis and including: (i) a proximal body end portion, and (ii) a distal body end portion; (b) a non-extensible core wire configured to inhibit longitudinal elongation of the helical wire coil body along the longitudinal coil axis; (c) a navigation sensor positioned within a distal body end portion of the helical coil body; and (d) a non-extensible tether connected between the core wire and the navigation sensor and configured to further inhibit longitudinal elongation of the helical wire coil body along the longitudinal coil axis between the core wire and the navigation sensor.

VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a helical wire coil body extending along a longitudinal coil axis and including:
(i) a proximal body end portion, and
(ii) a distal body end portion; and
(b) a non-extensible, core wire assembly configured to inhibit longitudinal elongation of the helical wire coil body along the longitudinal coil axis, wherein the core wire assembly includes:
(i) a first core wire distally extending from the proximal body end portion toward the distal body end portion, and
(ii) a second core wire distally extending from the proximal body end portion toward the distal body end portion and proximally terminating relative to the first core wire such that the distal body end portion is more flexible than the proximal body end portion,
wherein the first core wire and the second core wire transversely overlap to provide a collective column strength to the helical wire coil body along the longitudinal coil axis.

2. The apparatus of claim 1, wherein the helical wire coil body further includes an intermediate body portion extending between the proximal and distal body end portions, wherein the core wire assembly further includes a third core wire distally extending from the proximal body end portion toward the distal body end portion and proximally terminating relative to the second core wire such that the intermediate body portion is more flexible than the proximal body end portion, and wherein the first core wire, the second core wire, and the third core wire transversely overlap to provide the collective column strength to the helical wire coil body along the longitudinal coil axis.

3. The apparatus of claim 2, wherein the intermediate body portion is less flexible than the distal body end portion.

4. The apparatus of claim 3, wherein the first core wire terminates at a first distal wire end positioned within the distal body end portion, and wherein the second core wire terminates at a second distal wire end positioned within the intermediate body portion.

5. The apparatus of claim 4, wherein the third core wire terminates at a third distal wire end positioned within the proximal body end portion.

6. The apparatus of claim 5, wherein the second distal wire end proximally terminates from the first distal wire end with a distal length therebetween, and wherein the distal length therebetween is approximately 0.8 inches.

7. The apparatus of claim 6, wherein the third distal wire end proximally terminates from the first distal wire end with a proximal length therebetween, and wherein the proximal length therebetween is approximately 1.5 inches.

8. The apparatus of claim 1, wherein the second core wire is transversely secured relative to the first core wire by a wire bundle securement.

9. The apparatus of claim 8, wherein the wire bundle securement comprises an overmolding.

10. The apparatus of claim 8, wherein the core wire assembly has a distal tip, wherein the distal tip of the core wire assembly is secured to the distal body end portion of the helical wire coil body by a distal securement.

11. The apparatus of claim 1, wherein the second core wire extends in parallel with the first core wire.

12. The apparatus of claim 11, wherein the second core wire is positioned against the first core wire.

13. The apparatus of claim 1, further comprising a navigation sensor and a non-extensible tether, wherein the distal body end portion of the helical wire coil body contains the navigation sensor therein, and wherein the tether extends from the core wire assembly to the navigation sensor and is configured to inhibit elongation of the navigation sensor relative to the core wire assembly.

14. The apparatus of claim 1, wherein the helical wire coil body further includes:
(i) a proximal wire coil, wherein the proximal wire coil is helical, and
(ii) a distal wire coil, wherein the distal wire coil is helical and interlocked with the proximal wire coil such that the proximal and distal wire coils form a double helix configuration extending along the longitudinal coil axis.

15. The apparatus of claim 1, further comprising:
(a) a body;
(b) a guide extending distally from the body;
(c) a guidewire including the helical wire coil and the core wire assembly, wherein the guidewire is slidably disposed relative to the guide; and
(d) a dilation catheter slidably disposed relative to the guidewire, wherein the dilation catheter includes an expandable dilator.

16. An apparatus, comprising:
(a) a helical wire coil body extending along a longitudinal coil axis and including:
(i) a proximal body end portion,
(ii) a distal body end portion having:
(A) a proximal wire coil, wherein the proximal wire coil is helical, and
(B) a distal wire coil, wherein the distal wire coil is helical and interlocked with the proximal wire coil such that the proximal and distal wire coils form a double helix configuration extending along the longitudinal coil axis, and
(C) an intermediate body portion extending between the proximal and distal body end portions; and
(b) a non-extensible, core wire assembly configured to inhibit longitudinal elongation of the helical wire coil body along the longitudinal coil axis, wherein the core wire assembly includes:
(i) a first core wire distally extending from the proximal body end portion toward the distal body end portion,
(ii) a second core wire distally extending from the proximal body end portion toward the distal body end portion and proximally terminating relative to the first core wire such that the distal body end portion is more flexible than the proximal body end portion, and
(iii) a third core wire distally extending from the proximal body end portion toward the distal body end portion and proximally terminating relative to the second core wire such that the intermediate body portion is more flexible than the proximal body end portion,
wherein the first core wire, the second core wire, and the third core wire extend in parallel with each other and transversely overlap to provide a collective column strength to the helical wire coil body along the longitudinal coil axis.

17. The apparatus of claim 16, wherein the second core wire and the third core wire are transversely secured relative to the first core wire by a wire bundle securement.

18. The apparatus of claim 17, wherein the second core wire and the third core wire are each respectively positioned against the first core wire.

19. A method of manufacturing a guidewire, the method comprising:
(a) securing a first core wire having a first wire length relative to a second core wire having a second wire length to form a non-extensible, core wire assembly, wherein the first wire length is longer than the second wire length;
(b) inserting the core wire assembly through a helical wire coil body, wherein the helical wire coil body has a proximal body end portion and a distal body end portion and extends along a longitudinal coil axis; and
(c) securing the core wire assembly within the helical wire coil body such that the helical wire coil body is non-extensible with a collective column strength along the longitudinal coil axis and the distal body end portion is more flexible than the proximal body end portion.

20. The method of claim 19, wherein securing the first core wire having the first wire length relative to the second core wire having the second wire length further includes securing the first core wire relative to third second core wire having a third wire length to form the core wire assembly.
